(19) United States
(12) Patent Application Publication
(10) Pub. No.: US 2026/0137272 A1
(43) Pub. Date: May 21, 2026

(54) MULTI-CAMERA SYSTEM FOR DESCRIBING THE TEAR FILM OF AN EYE

(71) Applicant: Alcon Inc., Fribourg (CH)

(72) Inventors: Martin Gruendig, Rangsdorf (DE); Peter Zieger, Berlin (DE); Armin Haupt, Berlin (DE)

(21) Appl. No.: 19/381,395

(22) Filed: Nov. 6, 2025

Related U.S. Application Data
(60) Provisional application No. 63/722,668, filed on Nov. 20, 2024.

(51) Int. Cl. A61B 3/10; A61B 3/00; A61B 3/14

(52) U.S. Cl. CPC A61B 3/101; A61B 3/0041; A61B 3/14

(57) ABSTRACT

In certain embodiments, an ophthalmic system describes the tear film of an eye. A pattern illuminator directs a pattern of light towards the anterior surface of the eye, which reflects the pattern of light. Cameras (including an off-axis camera) generate images of the reflected pattern. A computer performs the following for multiple iterations to yield descriptor sets of the tear film: receive the images of the reflected pattern; determine a tear film value for each image point of each image to yield tear film values for each surface point of the eye; determine a tear film descriptor from the tear film values for each surface point to yield tear film descriptors for the surface points; and generate a descriptor set from the tear film descriptors of the surface points of the eye. The computer generates a tear film description using the descriptor sets.

### FIELD

Embodiments of the present disclosure relate to a multi-camera system for describing the tear film of an eye.

### BACKGROUND

Reflection-based diagnostic systems direct a pattern of light towards an eye, which reflects the light. For example, a Placido disc system directs light in a Placido disc pattern. The reflected light is analyzed to describe a feature of the eye, such as the anterior surface of the eye or the tear film of the eye.

### SUMMARY

In certain embodiments, an ophthalmic system for describing the tear film of an eye includes a pattern illuminator, cameras, and a computer. The pattern illuminator directs a pattern of light towards the anterior surface of the eye, which reflects the pattern of light as a reflected pattern. The anterior surface has surface points. The cameras include at least a first camera and a second camera, where the second camera comprises an off-axis camera. The first and the second cameras generate respective first and second images of the reflected pattern. The first image includes first image points corresponding to one or more surface points of the anterior surface, and the second image includes second image points corresponding to the one or more surface points of the anterior surface. The computer performs the following for at least two iterations to yield descriptor sets of the tear film generated during a sequence of times: receive the first and the second images of the reflected pattern from the first and the second cameras generated at a given time of the sequence of times; for the first image at the given time, determine first tear film values for the first image points to yield a first plurality of tear film values for the surface points; for the second image at the given time, determine second tear film values for the second image points to yield a second plurality of tear film values for the surface points; for each surface point, determine a tear film descriptor of the tear film at the surface point from at least the first tear film value and the second tear film value for the surface point, to yield tear film descriptors for the surface points; and generate a descriptor set that describes the tear film from the tear film descriptors of the surface points of the anterior surface at the given time. The computer generates a tear film description according to the descriptor sets generated during the sequence of times.

Embodiments may include one, more than one, any combination of, or all of the following:

For the first image at the given time, the computer determines the first tear film values for the first image points by: for each first image point, detecting a distortion of the reflected pattern indicating an irregularity of the tear film at the first image point; and calculating a tear film value for the first image point indicating the irregularity of the tear film at the first image point.

For each surface point, the computer determines the tear film descriptor of the tear film at the surface point by determining the tear film descriptor of the tear film at the surface point according to a function of at least the first tear film value and the second tear film value for the surface point.

For each surface point, the computer determines the tear film descriptor of the tear film at the surface point by determining the tear film descriptor of the tear film at the surface point according to an average of at least the first tear film value and the second tear film value for the surface point.

For each surface point, the computer determines the tear film descriptor of the tear film at the surface point by determining the tear film descriptor of the tear film at the surface point according to a weighted average of at least the first tear film value and the second tear film value for the surface point.

The computer generates the tear film description according to the curvature of the tear film-air interface of the tear film by determining the curvature of the tear film-air interface of the tear film according to the descriptor sets of the tear film; and identifying a tear film irregularity of the tear film from a curvature irregularity of the curvature of the tear film-air interface of the tear film.

The computer displays the tear film description via a display.

The computer generates the tear film description according to the descriptor sets generated during the sequence of times by generating one or more tear film graphics according to the descriptor sets. A tear film graphic describes an irregularity of the tear film.

The computer generates the tear film description according to the descriptor sets generated during the sequence of times by generating one or more tear film graphics according to the descriptor sets. A tear film graphic describes an irregularity of the tear film and comprises an overlay indicating the irregularity of the tear film.

The computer generates the tear film description according to the descriptor sets generated during the sequence of times by generating tear film graphics according to the descriptor sets. A tear film describing an irregularity of the tear film at a time of the sequence of times.

The computer generates the tear film description according to the descriptor sets generated during the sequence of times by generating a summary tear film graphic according to the descriptor sets. The summary tear film graphic describes the tear film during at least a subset of the sequence of times.

The computer generates the tear film description according to the descriptor sets generated during the sequence of times by generating a summary tear film graphic according to the descriptor sets. The summary tear film graphic describes an initial breakup time of the tear film at one or more locations of the anterior surface. The computer may generate the summary tear film graphic by: detecting an increase in a distortion of the reflected pattern; detecting a plateau in the distortion of the reflected pattern that occurs after the increase; and determining the initial breakup time of the tear film according to the increase and the plateau.

The computer generates the tear film description according to the descriptor sets generated during the sequence of times by describing a spread of a breakup of the tear film at one or more locations of the anterior surface according to the descriptor sets.

The ophthalmic system of Claim 1, the computer configured to perform the following during each iteration of the at least two iterations: generate a camera image from the first and the second images of the reflected pattern at each iteration, and display the camera image via a display.

The ophthalmic system of Claim 1, the computer configured to perform the following during each iteration of the at least two iterations: generate a camera image from the first and the second images of the reflected pattern at each iteration, generate an overlay describing the tear film from the descriptor set generated at each iteration, and display the camera image with the overlay.

The pattern comprises a Placido pattern comprising concentric rings.

The pattern comprises a Placido pattern comprising concentric rings and spots disposed between at least two adjacent concentric rings.

The pattern comprises a Placido pattern comprising concentric rings, where a concentric ring has a distinguishing feature that distinguishes the concentric ring from an adjacent concentric ring.

### BRIEF DESCRIPTION OF THE DRAWINGS

FIG. 1 illustrates an example of a multi-camera system that may be used to generate images of the tear film of the anterior surface of an eye, according to at least one embodiment described in the present disclosure;

FIG. 2 illustrates a top view of examples of a pattern illuminator and a camera system of a multi-camera system, according to at least one embodiment described in the present disclosure;

FIG. 3 illustrates an example of a computer system, according to at least one embodiment described in the present disclosure;

FIGS. 4 and 5 illustrate examples of cameras and images provided by the cameras, according to at least one embodiment described in the present disclosure;

FIG. 6 illustrates examples of images that may be used to perform a tear film analysis, according to at least one embodiment described in the present disclosure;

FIGS. 7A to 7D illustrate examples of camera images of a ring pattern taken over a period of time to detect changes in the tear film of an eye, according to at least one embodiment described in the present disclosure;

FIGS. 8A and 8B illustrate an example of using a graphic to describe a tear film, according to at least one embodiment described in the present disclosure; and

FIG. 9 illustrates an example of a method of generating a description of the tear film of an eye, according to at least one embodiment described in the present disclosure.

### DESCRIPTION OF EXAMPLE EMBODIMENTS

Referring now to the description and drawings, one or more example embodiments of the disclosed apparatuses, systems, and methods are shown in detail. The description and drawings are not intended to be exhaustive or otherwise limit the claims to the specific embodiments shown in the drawings and disclosed in the description. Although the drawings represent possible embodiments, the drawings are not necessarily to scale and certain features may be simplified, exaggerated, removed, or partially sectioned to better illustrate the embodiments.

Known reflection-based systems include an illuminator that provides patterned light and an on-axis camera located on the same axis as the illuminator. These reflection-based systems analyze the reflected patterned light to describe a feature of the eye, such as the tear film of the eye. The on-axis camera, however, blocks part of the light, causing a blind spot that yields a gap in the reflected pattern. While certain reflection-based systems attempt to reduce the gap, not all are successful. Moreover, the single on-axis camera may not provide adequate coverage or redundancy.

The present disclosure relates to a reflection-based system with multiple cameras. In certain embodiments, the cameras include an on-axis camera and at least one off-axis camera that generate images of the reflected patterned light from different, overlapping parts of the surface of the eye. The images can be registered with each other and used to describe a feature, e.g., the tear film, of the eye.

Certain embodiments of the present disclosure may provide improvements over known iterations of reflection-based systems. For example, the off-axis camera may detect reflections at the gap created by the on-axis camera. As another example, the multiple cameras may provide greater surface coverage and/or redundancy compared to systems with only one on-axis camera. As yet another example, an embodiment may analyze images of the reflected light to generate a description of the tear film of an eye. As yet another example, an embodiment may generate a description that shows the changes of the tear film over time.

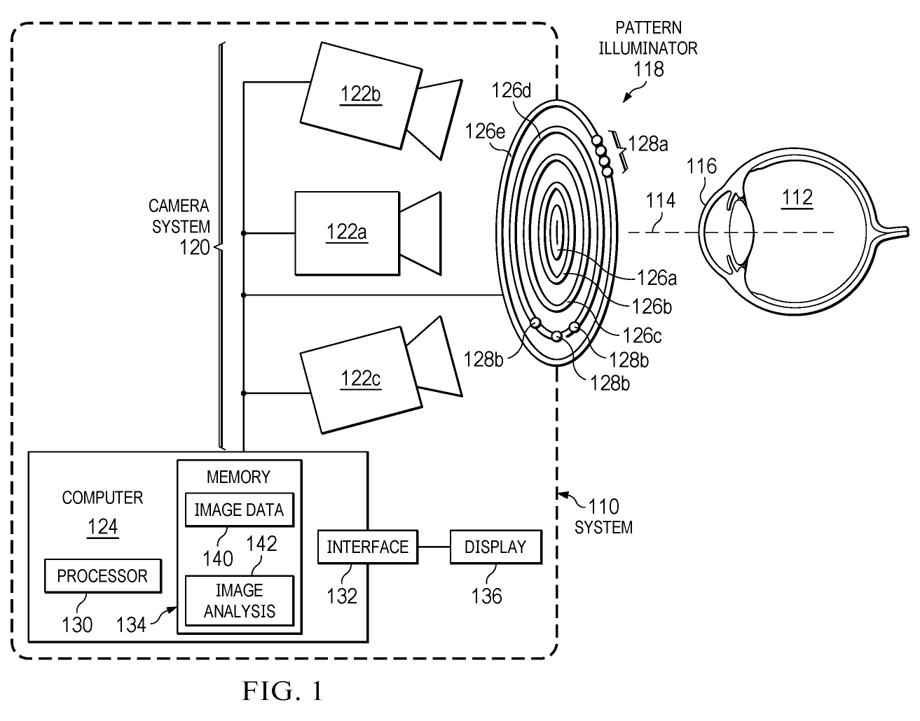

FIG. 1 illustrates an example of a multi-camera system 110 that may be used to generate images of the tear film of the anterior surface 116 of an eye 112, according to at least one embodiment described in the present disclosure. In the example, the system 110 includes a pattern illuminator 118, a camera system 120, and a computer 124. The camera system 120 comprises cameras 122 (e.g., 122a, 122b, and/or 122c), where the camera 122a is an on-axis camera and the cameras 122b and 122c are off-axis cameras. The camera 122a may be an on-axis camera that has an optical axis designed to align with an axis 114 (e.g., optical or visual) of the eye 112 when the eye 112 is properly aligned. The illuminator 118 includes illuminator rings 126 (e.g., 126a to 126e), which may have illuminator elements 128 (e.g., 128a and 128b). In the example, the illuminator elements 128a may yield, e.g., a circular shape comprising a solid line, and the illuminator elements 128b may yield, e.g., a circular shape comprising dots. The computer 124 includes one or more processors 130, an interface 132, a memory 134, and a display 136. The memory 134 stores data (e.g., image data 140) and applications (e.g., image analysis software 142).

As an example of an overview of operation, the pattern illuminator 118 directs a pattern of light towards the anterior surface of the eye 112, which reflects the pattern of light. The cameras 122a, 122b, 122c generate images of the reflected pattern, which can be analyzed to describe a feature, e.g., the tear film, of the eye 112. Each image has image points that represent surface points of the anterior surface. The computer 124 performs the following for multiple iterations to yield multiple descriptor sets of the tear film generated during a sequence of times: receives images of the reflected pattern from the cameras 122a, 122b, 122c; for each image, determines a tear film value for each image point of the image to yield tear film values describing the tear film at each surface point; for each surface point, determines a tear film descriptor of the tear film at the surface point from the tear film values of the surface point; and generates a descriptor set from the tear film descriptors of the surface points of the anterior surface. The computer 124 generates a tear film description (which may include one or more tear film graphics) from the descriptor sets and provides the tear film description to the display 136.

In the example, the eye 112 is the target of the system 110. The eye has an axis 114, which may be the visual and/or optical axis of the eye 112. The system 110 can provide descriptions of a feature of the anterior surface 116 of the eye 112, e.g., the tear film of the eye 112. The tear film of the eye 112 typically includes a mucin layer, an aqueous layer, and a lipid layer. The tear film may have an irregularity, e.g., where the tear film has an instability, has a discontinuity, has broken up, and/or does not provide adequate or sufficient coverage. A normal tear film without irregularities may be, e.g., approximately 1.3 to 3.7 microns thick. A tear film with an irregularity may be, e.g., less than 1.3 microns thick. The tear film-air interface reflects light, e.g., a pattern of light emitted by the pattern illuminator 118. The tear film-air interface has a curvature, which may provide information about the tear film.

Turning to the parts of the system 110, the pattern illuminator 118 directs light in a pattern (e.g., a Placido pattern) towards the anterior surface 116. The pattern may be any suitable pattern that can be reflected from the eye 112 to detect features of the surface of the eye 112. For example, the pattern may include one or more of the following: (1) a pattern (e.g., circular, polygonal, or rectangular array) of dots, dashes, and/or other figures, of the same or different sizes, equally or unequally spaced; (2) a series of lines (e.g., solid, dotted, and/or dashed), of the same or different thicknesses, equally or unequally spaced; and/or (3) concentric rings (e.g., solid, dotted, and/or dashed), of the same or different thicknesses, equally or unequally spaced.

As an example, the pattern may be a Placido pattern that has any suitable number of concentric rings, e.g., 3 to 15 concentric rings, such as 3 to 5, 5 to 10, and/or 10 to 15 concentric rings. As another example, any suitable number of spots (e.g., 4 to 50 spots, such as 4 to 10, 10 to 20, 20 to 30, 30 to 40, and/or 40 to 50 spots) may be disposed between adjacent rings of at least a subset of the concentric rings. As another example, a concentric ring may have a distinguishing feature that distinguishes the ring from an adjacent ring.

The pattern illuminator 118 may comprise any suitable arrangement of one or more light sources that can illuminate the anterior surface 116. In certain embodiments, the pattern illuminator 118 includes one or more illuminator rings 126. An illuminator ring 126 may provide any suitable circular pattern of light, e.g., solid, dotted, and/or dashed patterns. Different illuminator rings 126 may provide the same or different patterns. In certain embodiments, the pattern illuminator 118 includes illuminator elements 128, such as pixel illuminators, where each pixel illuminates the anterior surface 116 with a pixel of light. The pixels may be combined to yield the pattern. For example, the computer 124 may turn on and off particular pixel illuminators to yield a specific pattern.

In the example, the pattern illuminator 118 includes the illuminator rings 126a, 126c, and 126e that yield circular solid lines and the illuminator rings 126b and 125d that yield circular patterns of dots. The computer 124 may turn on most or all (80 to 100 %, such as 80 to 90 and/or 90 to 100 %) of the pixel illuminators of the illuminator rings 126a, 126c, and 126e to yield solid circular lines, and may turn on fewer (less than 25 %, such as 1 to 5, 5 to 10, and/or 10 to 25 %) of the pixel illuminators of the illuminator rings 126a, 126c, and 125e to yield the circular pattern of dots.

A camera 122 of the camera system 120 generates an image of the reflected pattern. A camera 122 may be any suitable camera that captures and records images. For example, a camera 122 may be a digital camera that records the image as digital image data. A digital camera 122 may include: an image sensor that detects light reflected from an object, such as a digital image sensor (e.g., CCD or CMOS); an image processor that converts the sensor output to digital image data representing the image; and a memory that records the image as image data 140.

The camera system 120 may have any suitable number (e.g., two, three, or more) and arrangement of cameras 122. In the example, the camera 122a is an on-axis camera, where the optical axis of the camera 122a substantially coincides with an axis 114 of the eye 112 when the eye 112 is in the proper position. The cameras 122b-c are off-axis cameras, where the optical axis of each camera 122b-c is at an angle with an axis of eye 112. The angle may have any suitable value, e.g., a value within the range of 1 to 10 degrees, 10 to 30 degrees, 30 to 40 degrees, or 40 to 60 degrees. The camera system 120 may include any suitable number of on-axis and/or off-axis cameras, and may include no on-axis cameras. In certain embodiments, the system 110 may include off-axis cameras that can be installed onto a system with existing on-axis instruments. In certain embodiments, system 110 may include only one camera 122 that moves to different positions (e.g., on-axis and/or off-axis) to operate as different cameras 122, such that the one camera is considered multiple cameras (e.g., by providing images from multiple positions).

The computer 124 generates a description of the tear film from images of a light pattern reflected from the eye 112. In certain embodiments, the computer 124 may perform the following for multiple iterations to yield multiple descriptor sets of a feature, such as the tear film, generated during a sequence of times: receives images of the reflected pattern from the cameras 122a, 122b, 122c; for each image, determines a tear film value for each image point of the image to yield tear film values describing the tear film at each surface point; for each surface point, determines a tear film descriptor of the tear film at the surface point from tear film values for the surface point; and generates a descriptor set from the tear film descriptors of the surface points of the anterior surface. The computer 124 generates a tear film description from the descriptor sets and provides the tear film description to the display 136.

In certain embodiments, the computer 124 determines tear film values for image points from the reflected pattern at the image points. A tear film value of an image point describes the tear film at the surface point represented by the image point, e.g., a tear film value may indicate an irregularity of the tear film at the surface point. In certain embodiments, the computer 124 calculates the tear film value of an image point in accordance with a distortion of the reflected pattern at the image point. A distortion of the reflected pattern may be a part of the pattern (e.g., line, dot, and/or other part of the pattern) that is not at the expected location, given the emitted pattern. A distortion may even cause a discontinuity in the pattern (e.g., a break in a line). A greater distortion (e.g., greater difference between the emitted pattern and the reflected pattern) may yield a tear film value indicating a greater irregularity of the tear film. In some examples, different ranges of distortions may yield different stages of tear film values. For example, a range of no or negligible distortions (e.g., less than 100 microns) may yield a normal tear film value representing normal tear film, and a range of larger distortions (e.g., greater than 100 microns) may yield an abnormal tear film value representing tear film with an irregularity, e.g., broken tear film.

In certain embodiments, the computer 124 calculates tear film descriptors. A tear film descriptor for a surface point is a combined tear film value calculated from multiple tear film values determined from multiple images of the surface point. A tear film descriptor may be calculated in any suitable manner. In certain embodiments, computer 124 determines the tear film descriptor at a surface point from a mathematical function, e.g., an average, of the one or more tear film values for the surface point. The average may be a weighted or unweighted average. For example, the average may be weighted by the quality of the image from each camera, where the value from a higher quality image has a greater weight than a value from a lower quality image. As another example, certain views may be weighted more heavily, such as images from the on-axis camera 122a being weighted more heavily than images from off-axis camera 122b, 122c. In certain embodiments, the computer 124 collects the tear film descriptors of the surface points of the anterior surface into a descriptor set.

The computer 124 generates a tear film description using the descriptor sets generated during the time period described by the sequence of times and may provide the tear film description to the display 136. The tear film description may include one or more tear film graphics, examples of which are described herein. A tear film graphic may be any suitable visual description of the tear film, e.g., a photo or video, a map, a chart, an overlay, or other visual description. In certain embodiments, the tear film description includes a tear film graphic describing an irregularity of the tear film at the surface points of the anterior surface. The tear film graphic may comprise images of the reflected image and an overlay indicating the irregularity of the tear film. For example, the overlay may indicate areas of normal, broken, and/or absent tear film.

In certain embodiments, the tear film description may describe the change in the tear film over time. For example, the computer 124 may generate a summary tear film graphic that describes the tear film over time. A summary tear film graphic may describe, e.g., an initial breakup time of the tear film at one or more locations of the anterior surface. As yet another example, the computer 124 may determine the spread of the breakup of the tear film across the anterior surface. In some embodiments, the computer 124 may generate a video as a sequence of depictions of the tear film over time, which may permit visualization of the progression of the breakup of the tear film.

In certain embodiments, the computer 124 calculates curvature values from the reflected pattern. The tear film-air interface that reflects the pattern may have a curvature, which may provide information about the tear film. In the embodiments, a curvature value for a surface point starts as a normal vector representing the surface normal at the surface point. The computer 124 determines whether there is a distortion in the reflected pattern and adjusts the normal vector according to the distortion. For example, if the distortion indicates a steeper curvature, the computer 124 adjusts the normal vector to represent the steeper curvature. If the distortion indicates a flatter curvature, the computer 124 adjusts the normal vector to represent the flatter curvature. An area of irregular (e.g., not smooth) curvature may indicate an irregularity of the tear film, such as broken tear film.

The curvature may be determined from the curvature values in any suitable manner. In certain embodiments, the computer 124 determines the curvature at a surface point from an average of the one or more curvature values for the surface point. The average may be a weighted or an unweighted average. For example, the average may be weighted by the quality of the image from each camera, where the value from a higher quality image has a greater weight than a value from a lower quality image.

The interface 132 and the memory 134 may be as described herein. The memory 134 stores data (e.g., image data 140) and applications (e.g., image analysis software 142). The image analysis software 142 processes the image data 140 to provide a description of the tear film. In certain embodiments, the image analysis software 142 registers the image data 140 from different cameras 122 to identify image points of different images that capture the same surface point. For example, the image analysis module 138 may analyze the image data 140 to identify landmark features of the eye 112, e.g., markings of an iris, that may be used to register images from different cameras. As another example, the image analysis module 138 may determine the field of view of each camera 122 from the arrangement of the cameras 122 and register the images based on the fields of view. In certain embodiments, the image analysis software 142 uses (e.g., merges, stitches, blends, selects from, or otherwise uses) one or more images from different cameras 122 to generate a single composite camera image representative of the eye 112.

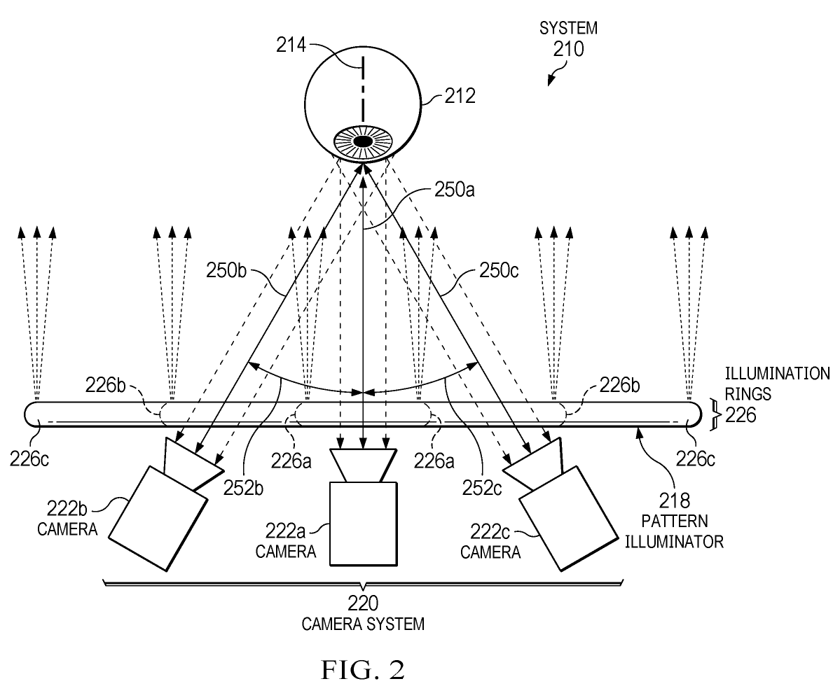

FIG. 2 illustrates a top view of examples of a pattern illuminator 218 and a camera system 220 of a multi-camera system 210, according to at least one embodiment described in the present disclosure. The pattern illuminator 218 includes concentric illumination rings 226 (226a to 226c) that direct a pattern of light towards an eye 212. The illumination ring 226a is the innermost ring, the illumination ring 226c is the outermost ring, and the illumination ring 226b is disposed between illumination ring 226a and illumination ring 226c.

The camera system 220 includes cameras 222a to 222c that generate images of the reflected pattern. The camera 222a is an on-axis camera designed to align with an axis 214 of an eye 212 when the patient is properly aligned relative to the system 210. The cameras 222b-c are off-axis cameras, where the optical axis of each camera 222b-c is at an angle with the axis 214, as described herein.

The cameras 222a to 222c are in a specific arrangement relative to a common focal point, which may be at the eye 212 when the patient is properly aligned. The camera 222a is at a distance 250a away from the surface of the eye 212, the camera 222b is at a distance 250b away from the surface, and the camera 222c is at a distance 250c away from the surface. The axis of the camera 222a is at an angle 252b away from the axis of camera 222a, the axis of the camera 222c is at an angle 252b away from the axis of camera 222c. In certain embodiments, the arrangement may assist a computer of the system 210 with registering one or more images from different cameras 222 to yield a camera image.

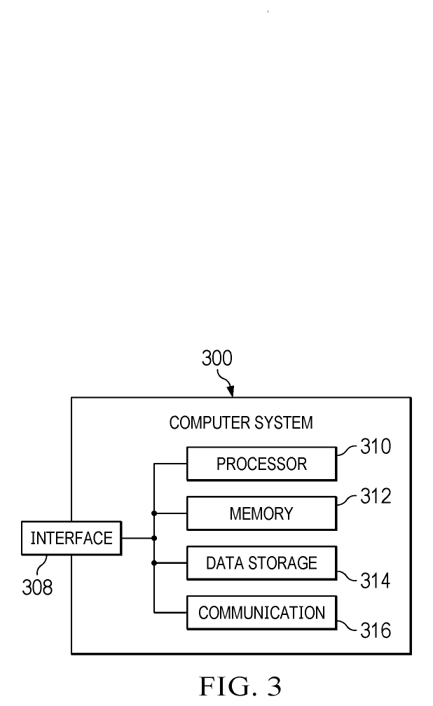

FIG. 3 illustrates an example of a computer system 300, according to at least one embodiment described in the present disclosure. The computer system 300 may include an interface 308, a processor 310, a memory 312, a data storage 314, and/or a communication subsystem 316, any or all of which may be communicatively coupled. Any or all of the computer system 300 may be implemented as computer hardware and/or software. Any or all of the computer systems described herein may be implemented as a computer system consistent with the computer system 300.

In the example, the interface 308 may receive input to the computer system 300 and/or send output from the computer system 300, and may be used to exchange information between, e.g., software, hardware, one or more peripheral devices, one or more users, and/or any suitable combinations of any of the preceding. A user interface is a type of interface that a user can utilize to communicate with (e.g., send input to and/or receive output from) the computer system 300. Examples of user interfaces include displays, Graphical User Interfaces (GUIs), touchscreens, foot pedals, keyboards, computer mouses (or mice), gesture sensors, microphones, and speakers.

Generally, the processor 310 may include any suitable special-purpose or general-purpose computer, computer entity, or processing device including various computer hardware or software modules and may be configured to execute instructions stored on any applicable computer-readable storage media. For example, the processor 310 may include a microprocessor, a microcontroller, a digital signal processor (DSP), an application-specific integrated circuit (ASIC), a Field-Programmable Gate Array (FPGA), or any other digital or analog circuitry configured to interpret and/or to execute program instructions and/or to process data. Although illustrated as a single processor in FIG. 3, the processor 310 may include any number of processors distributed across any number of network or physical locations that are configured to perform individually or collectively any number of operations described in the present disclosure.

The processor 310 may perform any suitable operations. In some embodiments, the processor 310 may interpret and/or execute program instructions and/or process data stored in the memory 312, the data storage 314, or the memory 312 and the data storage 314. In some embodiments, the processor 310 may fetch program instructions from the data storage 314 and load the program instructions into the memory 312. After the program instructions are loaded into the memory 312, the processor 310 may execute the program instructions, such as instructions to perform any of the methods disclosed herein, respectively.

The memory 312 and the data storage 314 may include computer-readable storage media or one or more computer-readable storage mediums for carrying or having computer-executable instructions or data structures stored thereon. Such computer-readable storage media may be any available media that may be accessed by a general-purpose or special-purpose computer, such as the processor 310.

By way of example, and not limitation, such computer-readable storage media may include non-transitory computer-readable storage media including Random Access Memory (RAM), Read-Only Memory (ROM), Electrically Erasable Programmable Read-Only Memory (EEPROM), Compact Disc Read-Only Memory (CD-ROM) or other optical disk storage, magnetic disk storage or other magnetic storage devices, flash memory devices (e.g., solid state memory devices), or any other storage medium which may be used to carry or store desired program code in the form of computer-executable instructions or data structures and which may be accessed by a general-purpose or special-purpose computer. Combinations of the above may also be included within the scope of computer-readable storage media. Computer-executable instructions may include, for example, instructions and data configured to cause the processor 310 to perform a certain operation or group of operations.

The communication subsystem 316 may include any component, device, system, or combination thereof that is configured to transmit, receive, and/or otherwise exchange information over a network in order to communicate with any suitable entity, such as with other devices at other locations or at the same location or even within the same system. The communication subsystem 316 may provide for communication among the devices described in the present disclosure, communication networks, computing devices, and other systems. For example, the communication subsystem 316 may allow the system 300 to communicate with other systems, such as other computing devices and/or networks. In some embodiments, the communication subsystem 316 may include a modem, a network card (wireless or wired), an optical communication device, an infrared communication device, a wireless communication device (such as an antenna), and/or chipset. Examples of communication subsystem 316 include a Bluetooth device, an 802.6 device (e.g., that can communicate with a Metropolitan Area Network (MAN)), a WiFi device, a WiMax device, cellular communication facilities, and/or the like.

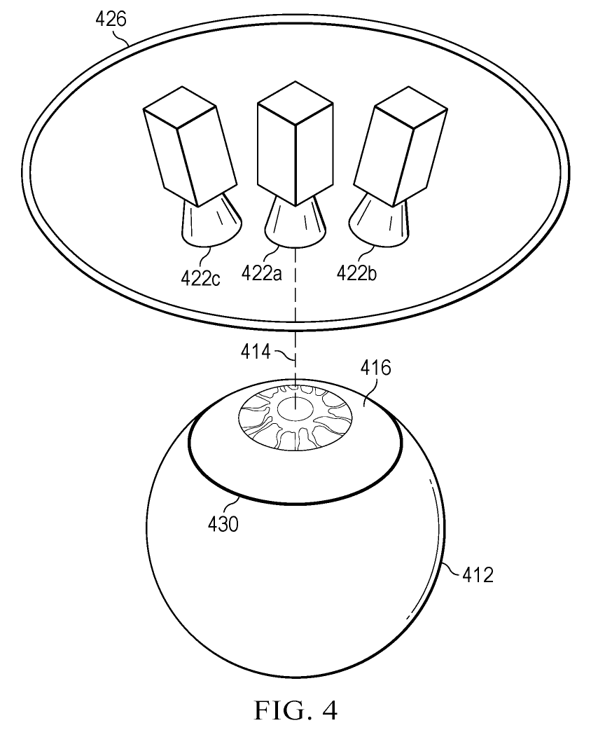

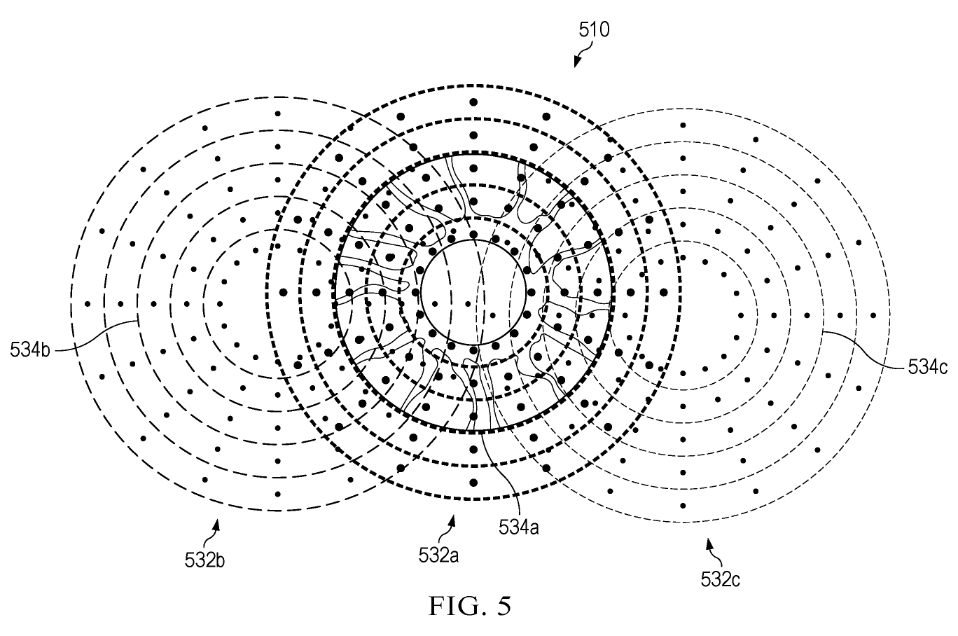

FIGS. 4 and 5 illustrate examples of cameras 422 (422a to 422c) and images 532 (523a to 532c) provided by the cameras 422 (422a to 422c, respectively), according to at least one embodiment described in the present disclosure. FIG. 4 illustrates examples of the cameras 422 (422a to 422c) and an illumination ring 426. The illumination ring 426 yields a ring 430 on the anterior surface 416 of the eye 412. For ease of explanation, only one illumination ring 426 that yields one ring 430 is shown. The cameras 422 (422a to 422c) provide image data describing the anterior surface 416 of the eye 412. The cameras 422 (422a to 422c) include an on-axis camera aligned with eye axis 414 and off-axis cameras 422b and 422c.

FIG. 5 illustrates an example of a camera image 510 that includes images 532 (532a to 532c) of a ring pattern captured by the cameras 422 (422a to 422c, respectively) of FIG. 4: the camera 422a provides the image 532a of the ring pattern; the camera 422b provides the image 532b of the ring pattern; and the camera 422c provides the image 532c of the ring pattern. The image 532a includes an image 534a of the ring 430 of FIG. 4. Similarly, the image 532b includes an image 534b of the ring 430 of FIG. 4, and the image 532c includes an image 534c of the ring 430 of FIG. 4.

The illustrated example of the cameras 422 may provide advantages. For example, the corneal coverage provided by multiple cameras 422 is greater than the coverage provided by one camera. Thus, the cameras 422 can describe a feature (e.g., the tear film) of a greater area of the anterior surface 416. As another example, portions of the anterior surface 416 are imaged by more than one camera. Thus, multiple cameras 422 provide redundancy that yields more reliable results. As yet another example, images from the cameras 422 describe pattern distortions in more directions than images from a single camera. Thus, the images from multiple cameras 422 may better detect distortions. As yet another example, the off-axis cameras 422b and 422c can detect aberrations at or near the axis 414 that the on-axis camera 422a might not be able to detect. Accordingly, the cameras 422 may provide improved image data for analyzing a feature of the anterior surface 416.

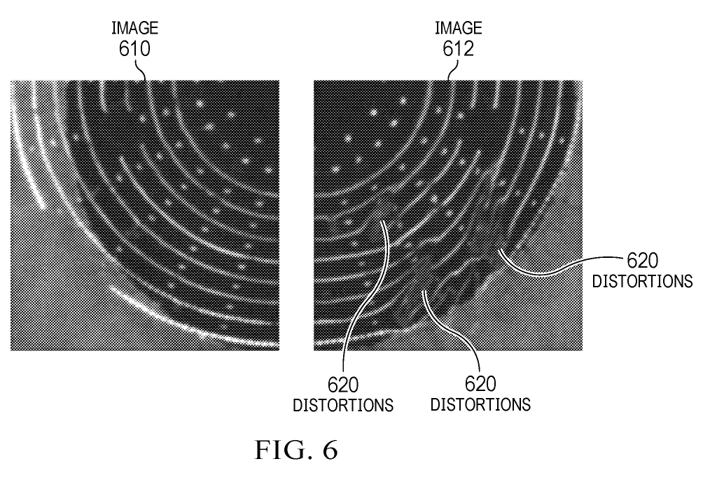

FIG. 6 illustrates examples of images 610 and 612 that may be used to perform a tear film analysis, according to at least one embodiment described in the present disclosure. Image 610 shows an intact pattern that indicates the tear film is a normal, substantially unbroken layer. Image 612 shows a distorted pattern with distortions 620 that indicate irregularities of the tear film, such as areas where the tear film is too thin (less than 1.3 microns) or essentially non-existent. As discussed herein, a distortion is a difference between the imaged pattern and the expected pattern, e.g., a part of the pattern that is not at the expected location, a discontinuity in the pattern, or other difference.

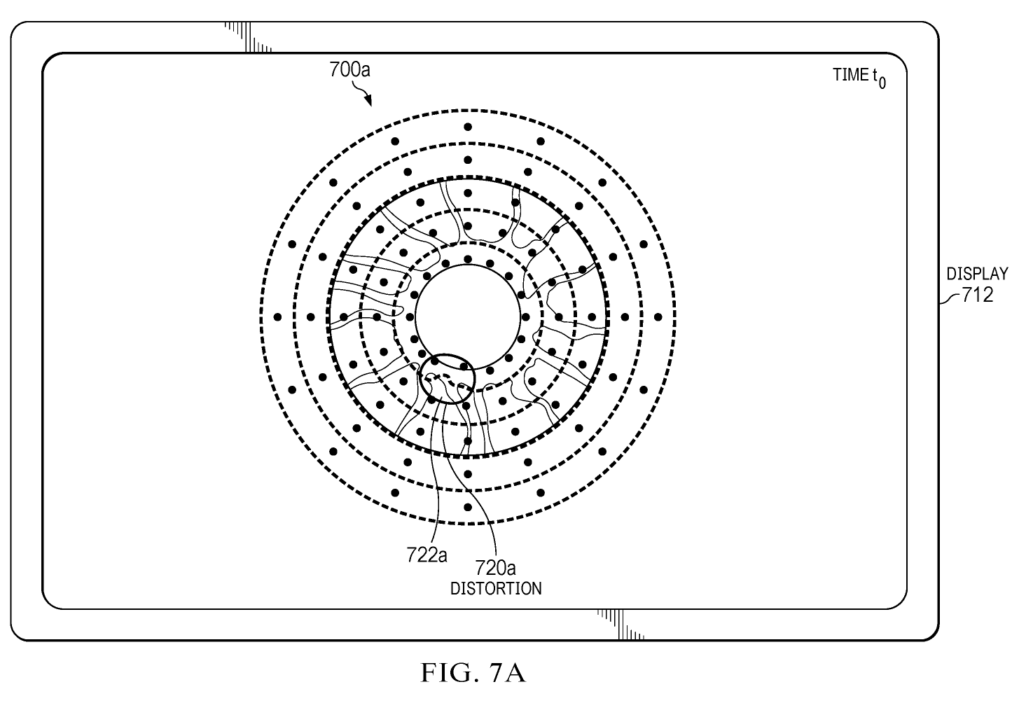

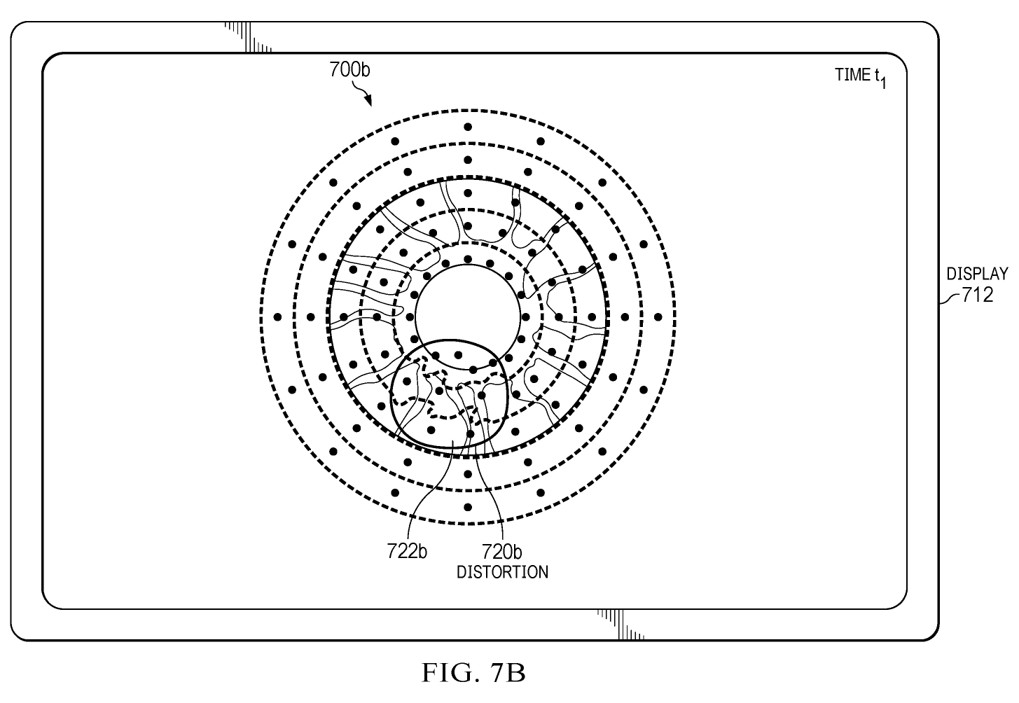

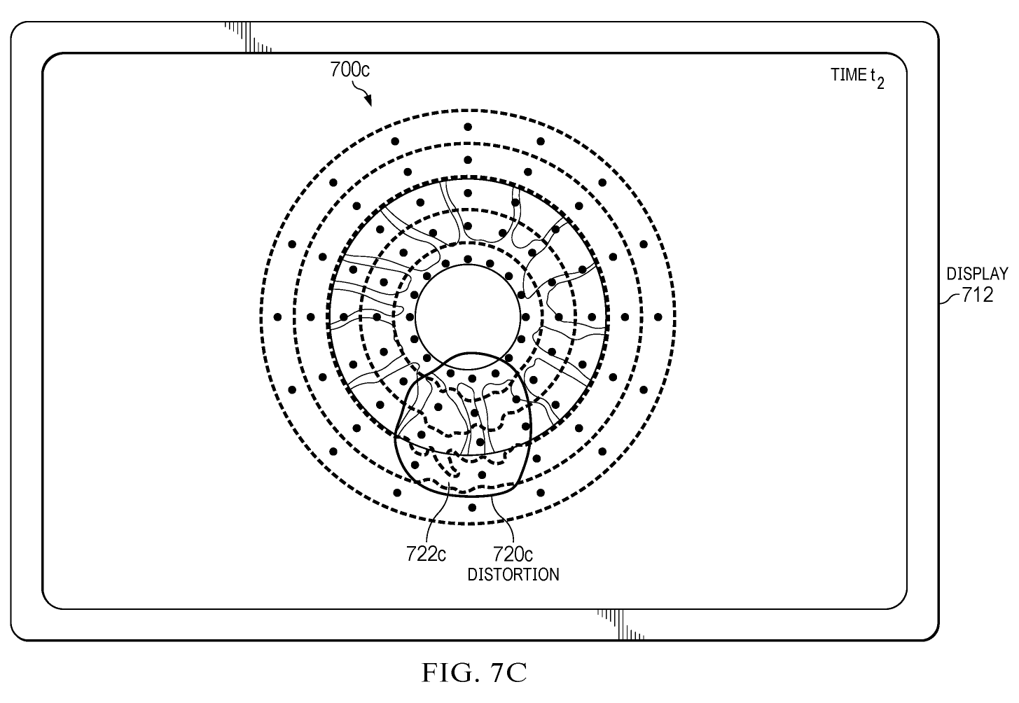

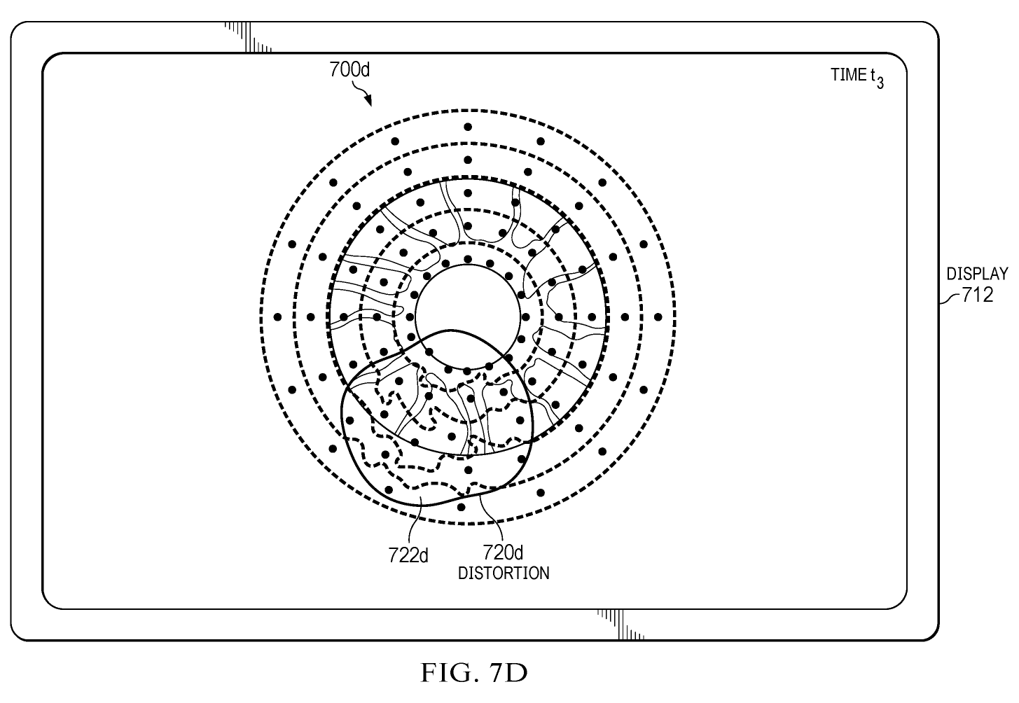

FIGS. 7A to 7D illustrate examples of camera images 700 (700a to 700d) of a ring pattern taken over a period of time to detect changes in the tear film of an eye, according to at least one embodiment described in the present disclosure. In the example, a display 712 presents the camera images 700 (700a to 700d). The camera images 700 (700a to 700d) indicate the status of tear film of the anterior surface of the eye over a period of time. Any suitable number of images (e.g., 2 to 10, 10 to 25, 25 or more), which may be provided in a video, may be taken over any suitable period of time (e.g., 0.5 to 2, 2 to 5, 5 to 10, and/or greater than 10 seconds). In the example, images are taken at time t0, time t1, time t2, and time t3.

In the examples, the camera images 700 (700a to 700d) show a distortion 720 (720a to 720d, respectively) that changes over time t0 to time t3. The distortion 720 (720a to 720d) indicates an irregularity 722 (722a to 722d, respectively) of the tear film, such as an area where the tear film is too thin or essentially non-existent, e.g., where the tear film is breaking up. At time t0, the distortion 720a is smaller and increases in size from time t1 to time t3.

The tear film irregularity 722 may be shown in an image in any suitable manner. For example, the irregularity 722 may be highlighted in one or more of the following ways: (a) outlined, e.g., as shown in the illustrated example; (b) displayed in a different color (which may be a color of the visible spectrum or greyscale); (c) overlayed with a pattern (e.g., an array of lines, dots, or other markings); and/or portrayed with any other suitable graphical technique. In some embodiments, the tear film irregularity 722 may be depicted as an overlay on the captured image (e.g., from the on-axis camera) and/or the composite image.

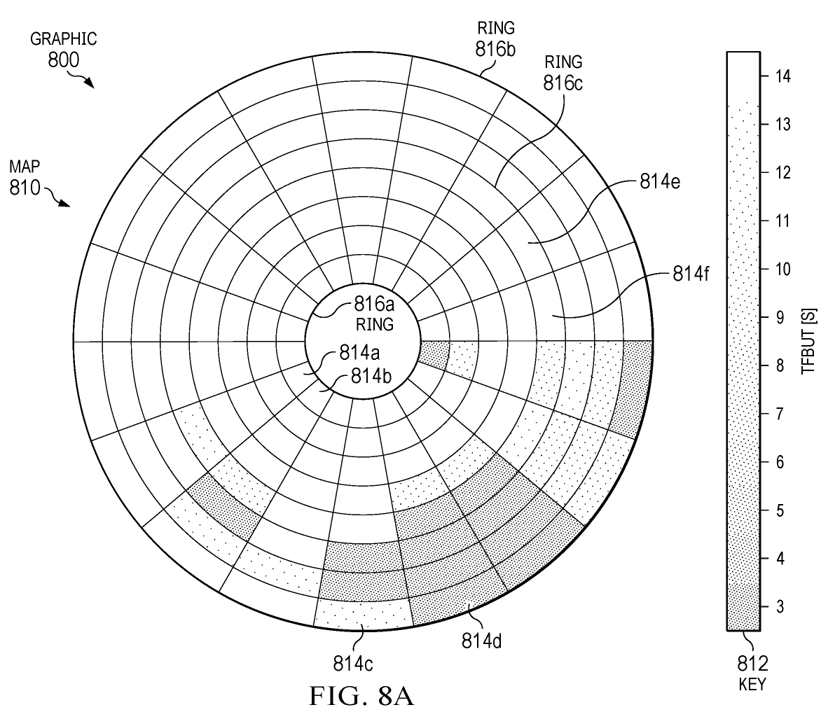

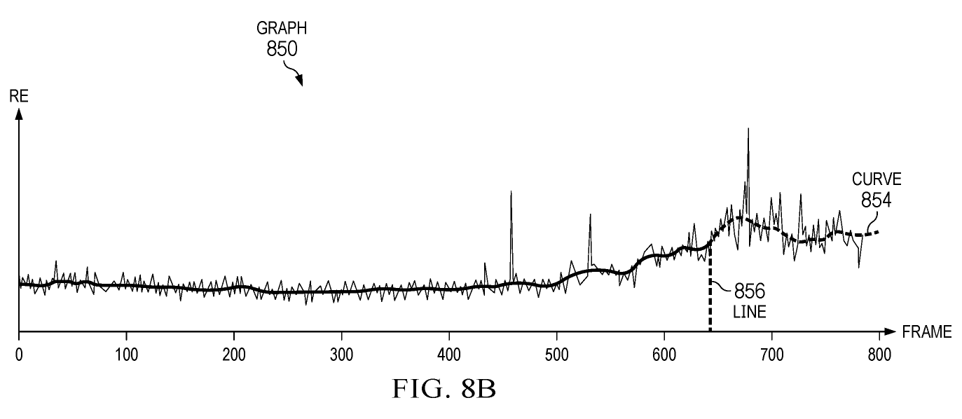

FIGS. 8A and 8B illustrate an example of using a graphic to describe a tear film, according to at least one embodiment described in the present disclosure. FIG. 8A illustrates an example of a tear film graphic 800. FIG. 8B illustrates an example of a tear film progression graph 850 that may be used to generate the tear film graphic 800.

FIG. 8A illustrates the example of the tear film graphic 800. The tear film graphic 800 includes a tear film map 810 and a map key 812. The tear film map 810 includes sectors 814 (e.g., 814a to 814f) that represent regions of the anterior surface of an eye. The tear film map 810 and sectors 814 may have any suitable shape, size, and/or appearance. In the example, the tear film map 810 includes rings 816 (e.g., 816a, 816b, and 816c) with sectors 814 (e.g., 814a to 814f). The rings 816 include an innermost ring 816a, an outermost ring 816b, and intermediate rings such as ring 816c. The sectors 814 are segments of their respective ring 816. For example, the ring 816a includes, e.g., the sectors 814a and 814b, as well as other sectors; the ring 816b includes, e.g., the sectors 814c and 814d, as well as other sectors; and the ring 816c includes, e.g., the sectors 814e and 814f, as well as other sectors. A sector 814 has a sector value, e.g., a color (such as greyscale value or color value), a pattern (examples described herein), one or more characters (e.g., alphanumeric characters), and/or other suitable value that can be representative of the sector 814. The map key 812 conveys the tear film description that the sector value represents.

In the illustrated example, the tear film graphic 800 is a summary tear film graphic that describes the tear film over time. In the example, the tear film map 810 shows the distribution of initial breakup times across the anterior surface, which also indicates the spread of the initial breakup times over time. The sector value is a pattern value that represents the time at which the breakup of the tear film initially occurred, i.e., the initial breakup time. The map key 812 provides the time (in seconds) of the initial breakup time. For example, for a given sector 814, the sector value may be the time at which the given sector 814 initially experienced a predetermined pattern of distortion indicative of a tear film breakup, as described in more detail below. Each of the sector values may then be compiled into the tear film graphic 800.

The tear film graphic 800 describes the tear film over time. For ease of explanation, locations of the tear film graphic 800 may correspond to locations of an analog clock face, where a direction of the tear film graphic 800 that starts at the center and moves upward corresponds to the 12 o’clock direction of the analog clock face, and a direction of the tear film graphic 800 that starts at the center and moves towards the right corresponds to the 3 o’clock direction of the analog clock face. According to the tear film graphic 800, the tear film began breaking up mainly in the lower region of the eye around the 4 o’clock and 5 o’clock position near the periphery of the eye more quickly than the upper region of the eye. The tear film continued to slowly break apart outwardly from the lower region up towards the 3 o’clock position and more towards the middle of the eye. The upper portions of the eye did not experience significant tear film breakup.

FIG. 8B illustrates the example of the tear film progression graph 850 that may be used to generate the tear film graphic 800. The tear film progression graph 850 describes how, for a particular sector 814, the tear film changes over time. The tear film progression graph 850 may describe the changes in any suitable manner that allows for determination of the initial breakup time of the sector 814. In the example, tear film progression graph 850 includes an x-axis that gives frame numbers for a sequence of frames and a y-axis that represents reconstruction error (RE) values. In the example, a curve 854 shows the pattern of distortion, and a vertical line 856 shows the initial breakup time. The sequence of frames may be the sequence of actual images of the sector 814 taken over time. The reconstruction error measures how much the actual image of the sector 814 deviates from an expected image of the sector 814, which may indicate when there is a distortion associated with a tear film breakup. The reconstruction error may be determined in any suitable manner. For example, a neural network (e.g., an autoencoder) may be used to measure the difference between the actual image and the expected image.

The curve 854 may indicate a pattern of distortion associated with a tear film breakup. When the tear film initially breaks up, the distortion increases, which may be indicated by, e.g., the increasing reconstruction error values around frames 640 to 675. When the tear film has evaporated, the distortion form a plateau, which may be indicated by, e.g., the substantially constant reconstruction error values after frame 700. Determining the initial breakup time according to the pattern of distortion over time may provide more reliable results. Using the pattern over time filters out uninformative, transient ripples that may occur during tear film buildup or may be caused by moving particles. Moreover, using the pattern over time does not rely on having specific starting reconstruction error values, as the change in the values over time is used to identify a tear film breakup.

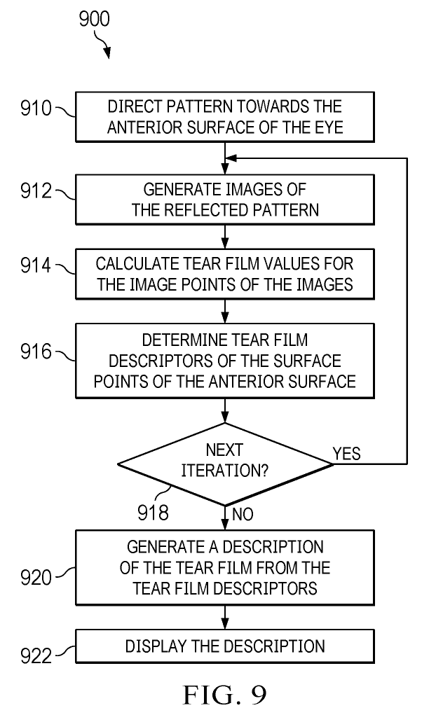

FIG. 9 illustrates an example of a method 900 of generating a description of the tear film of an eye, according to at least one embodiment described in the present disclosure. In certain embodiments, the method 900 may be performed for one or more iterations, where each iteration may describe the tear film at a particular time. For example, the method 900 may be performed for one iteration to describe the tear film at a particular time. As another example, the method 900 may be performed for more than one iteration to describe the tear film at more than one time in order to describe a change in the tear film over time.

At block 910, a patten illuminator directs a pattern towards the anterior surface of the eye. Any suitable pattern may be used, e.g., a Placido pattern. The anterior surface of the eye, which has a tear film, reflects the pattern.

At block 912, one or more cameras generate images of the reflected pattern. The images may overlap with each other.

At block 914, a computer calculates tear film values for the image points of the images. A tear film value for an image point may indicate whether there is an irregularity of the tear film at the image point.

At block 916, the computer determines tear film descriptors of the surface points of the anterior surface. The computer may apply a mathematical function (e.g., an average or a weighted average) to multiple tear film values at an image point to determine the tear film descriptor for the surface point corresponding to the image point. The tear film descriptor of a surface point may indicate whether there is an irregularity in the tear film at the surface point.

At block 918, there may be a next iteration to perform. If there is a next iteration, the method proceeds to block 912. If there is no next iteration, the method proceeds to block 920.

At block 920, the computer generates a description of the tear film from the tear film descriptors. The description may identify irregularities in the tear film at one or more times.

At block 922, the computer displays the description of the tear film. The description may highlight the irregularities of the tear film and/or may show the changes in the tear film over time.

The present disclosure (including the specification, claims, and drawings) includes example embodiments that are intended to aid the reader in understanding the invention and concepts contributed by the inventor to furthering the art and to enable any person skilled in the art to make or use the disclosed embodiments. Modifications (e.g., changes, substitutions, additions, omissions, and/or other modifications) to the embodiments will be readily apparent to those skilled in the art. Accordingly, modifications may be made to the embodiments without departing from the essence of the present disclosure.

In certain instances, modifications may be made to the systems disclosed herein, as apparent to those skilled in the art. For example, parts of a system may be integrated or separated, or an operation of a system may be performed by more, fewer, or other parts. In certain instances, modifications may be made to the methods disclosed herein, as apparent to those skilled in the art. For example, the methods may include more, fewer, or other operations. As another example, certain operations may be optional, combined into fewer operations, or expanded into additional operations. As yet another example, certain operations may be performed in any suitable order or simultaneously.

Furthermore, those skilled in the art will recognize that the present disclosure is not intended to be limited to the example embodiments and that the language of the disclosure is to be accorded the widest scope consistent with the present disclosure. Terms (which may include one or more words) that describe inclusion are generally intended as “open” terms in that they generally do not imply exclusion. For example, the term “including” may be interpreted as “including, but not limited to” or “including at least”; the term “having” may be interpreted as “having, but not limited to” or “having at least”; and the term “comprising” may be interpreted as “comprising, but not limited to” or “comprising at least”, etc.

Additionally, if a specific number is intended, such intent will be explicitly recited in the claim. In the absence of the explicit recitation of a specific number, no such intent is present. If a specific number is explicitly recited, such recitation should be interpreted to mean at least the recited number. For example, the bare recitation of “two Xs”, without other modifiers, may mean “at least two Xs” or “two or more Xs”. Moreover, the use of an indefinite article (e.g., “a” or “an”) or definite article (e.g., “the”) to introduce a noun phrase should not be construed to limit the noun phrase to one, but may be interpreted as an open term “at least one” or “one or more”. This holds even when the same claim includes an open term (e.g., “one or more” or “at least one”) and an indefinite or definite article (e.g., “a” or “an” or “the”).

Moreover, a selection from a list of items should be understood to contemplate a selection of any suitable individual item or any suitable combination of items. For example, the general construction “at least one of A, B, and C” or “one or more of A, B, and C” may include A alone; B alone; C alone; A and B together; A and C together; B and C together; and A, B, and C together. Moreover, any disjunctive term presenting two or more alternative items may be understood to contemplate including one of the items, either of the items, or both items. For example, the general construction “A or B” or “A and/or B” may include A alone, B alone, and A and B together. Additionally, the use of the terms “first,” “second,” “third,” etc. are not necessarily used herein to connote a specific order. For example, the terms “first,” “second,” “third,” etc., may be used to distinguish between different elements.

To aid the Patent Office and readers in interpreting the claims, Applicants note that they do not intend any of the claims or claim elements to invoke 35 U.S.C. §112(f), unless the words “means for” or “step for” are explicitly used in the particular claim. Use of any other term (e.g., “mechanism,” “module,” “device,” “unit,” “component,” “element,” “member,” “apparatus,” “machine,” “system,” “processor,” or “controller”) within a claim is understood by the Applicants to refer to structures known to those skilled in the art and is not intended to invoke 35 U.S.C. §112(f).

What is claimed:

1. An ophthalmic system for describing a tear film of an eye, the ophthalmic system comprising:
a pattern illuminator configured to direct a pattern of light towards an anterior surface of the eye, the anterior surface having a plurality of surface points, the anterior surface reflecting the pattern of light as a reflected pattern;
at least a first camera and a second camera, the first and the second cameras configured to generate respective first and second images of the reflected pattern, the second camera comprising an off-axis camera, the first image including a plurality of first image points corresponding to one or more surface points of the plurality of surface points of the anterior surface and the second image including a plurality of second image points corresponding to the one or more surface points of the plurality of surface points of the anterior surface; and
a computer configured to:
perform the following for at least two iterations to yield a plurality of descriptor sets of the tear film generated during a sequence of times:
receive the first and the second images of the reflected pattern from the first and the second cameras generated at a given time of the sequence of times;
for the first image at the given time, determine a plurality of first tear film values for the first image points to yield a first plurality of tear film values for the plurality of surface points;
for the second image at the given time, determine a plurality of second tear film values for the second image points to yield a second plurality of tear film values for the plurality of surface points;
for each surface point of the plurality of surface points, determine a tear film descriptor of the tear film at the each surface point from at least the first tear film value and the second tear film value for the each surface point, to yield a plurality of tear film descriptors for the plurality of surface points; and
generate a descriptor set that describes the tear film from the plurality of tear film descriptors of the plurality of the surface points of the anterior surface at the given time; and
generate a tear film description according to the plurality of descriptor sets generated during the sequence of times.

2. The ophthalmic system of claim 1, the computer configured to, for the first image at the given time, determine the plurality of first tear film values for the first image points to yield the first plurality of tear film values for the plurality of surface points by:
for each first image point of the plurality of first image points of the respective image, detecting a distortion of the reflected pattern indicating an irregularity of the tear film at the first image point; and
calculating a tear film value for the first image point indicating the irregularity of the tear film at the first image point.

3. The ophthalmic system of claim 1, the computer configured to, for each surface point of the plurality of surface points, determine the tear film descriptor of the tear film at the each surface point from at least the first tear film value and the second tear film value for the each surface point by:
for the each surface point, determining the tear film descriptor of the tear film at the each surface point according to a function of at least the first tear film value and the second tear film value for the each surface point.

4. The ophthalmic system of claim 1, the computer configured to, for each surface point of the plurality of surface points, determine the tear film descriptor of the tear film at the each surface point from at least the first tear film value and the second tear film value for the each surface point by:
for the each surface point, determining the tear film descriptor of the tear film at the each surface point according to an average of at least the first tear film value and the second tear film value for the each surface point.

5. The ophthalmic system of claim 1, the computer configured to, for each surface point of the plurality of surface points, determine the tear film descriptor of the tear film at the each surface point from at least the first tear film value and the second tear film value for the each surface point by:
for the each surface point, determining the tear film descriptor of the tear film at the each surface point according to a weighted average of at least the first tear film value and the second tear film value for the each surface point.

6. The ophthalmic system of claim 1, the computer further configured to generate the tear film description according to a curvature of a tear film-air interface of the tear film by:
determining the curvature of the tear film-air interface of the tear film according to the plurality of descriptor sets of the tear film; and
identifying a tear film irregularity of the tear film from a curvature irregularity of the curvature of the tear film-air interface of the tear film.

7. The ophthalmic system of claim 1, the computer configured to:
display the tear film description via a display.

8. The ophthalmic system of claim 1, the computer configured to generate the tear film description according to the plurality of descriptor sets generated during the sequence of times by:
generating one or more tear film graphics according to the plurality of descriptor sets, a tear film graphic of the one or more tear film graphics describing an irregularity of the tear film.

9. The ophthalmic system of claim 1, the computer configured to generate the tear film description according to the plurality of descriptor sets generated during the sequence of times by:
generating one or more tear film graphics according to the plurality of descriptor sets, a tear film graphic of the one or more tear film graphics describing an irregularity of the tear film, the tear film graphic comprising an overlay indicating the irregularity of the tear film.

10. The ophthalmic system of claim 1, the computer configured to generate the tear film description according to the plurality of descriptor sets generated during the sequence of times by:
generating a plurality of tear film graphics according to the plurality of descriptor sets, a tear film graphic of the plurality of tear film graphics describing an irregularity of the tear film at a time of the sequence of times.

11. The ophthalmic system of claim 1, the computer configured to generate the tear film description according to the plurality of descriptor sets generated during the sequence of times by:
generating a summary tear film graphic according to the plurality of descriptor sets, the summary tear film graphic describing the tear film during at least a subset of the sequence of times.

12. The ophthalmic system of claim 1, the computer configured to generate the tear film description according to the plurality of descriptor sets generated during the sequence of times by:
generating a summary tear film graphic according to the plurality of descriptor sets, the summary tear film graphic describing an initial breakup time of the tear film at one or more locations of the anterior surface.

13. The ophthalmic system of claim 12, the computer configured to generate the summary tear film graphic according to the plurality of descriptor sets by:
detecting an increase in a distortion of the reflected pattern;
detecting a plateau of the distortion of the reflected pattern that occurs after the increase in the distortion of the reflected pattern; and
determining the initial breakup time of the tear film according to the increase in the distortion of the reflected pattern and the plateau of the distortion of the reflected pattern.

14. The ophthalmic system of claim 1, the computer configured to generate the tear film description according to the plurality of descriptor sets generated during the sequence of times by:
describing a spread of a breakup of the tear film at one or more locations of the anterior surface according to the plurality of descriptor sets.

15. The ophthalmic system of claim 1, the computer configured to perform the following during each iteration of the at least two iterations:
generate a camera image from the first and the second images of the reflected pattern at the each iteration; and
display the camera image via a display.

16. The ophthalmic system of claim 1, the computer configured to perform the following during each iteration of the at least two iterations:
generate a camera image from the first and the second images of the reflected pattern at the each iteration;
generate an overlay describing the tear film from the descriptor set generated at the each iteration; and
display the camera image with the overlay.

17. The ophthalmic system of claim 1, the pattern comprising a Placido pattern comprising a plurality of concentric rings.

18. The ophthalmic system of claim 1, the pattern comprising a Placido pattern comprising:
a plurality of concentric rings; and
a plurality of spots disposed between at least two adjacent concentric rings of the plurality of concentric rings.

19. The ophthalmic system of claim 1, the pattern comprising a Placido pattern comprising a plurality of concentric rings, a concentric ring of the plurality of concentric rings having a distinguishing feature that distinguishes the concentric ring from an adjacent concentric ring.
